(19) United States
(12) Patent Application Publication
(10) Pub. No.: US 2008/0085331 A1
(43) Pub. Date: Apr. 10, 2008

(54) Composition and method for raising blood glucose level

(76) Inventors: Anna E. Gluskin, Toronto (CA); Muhammad Waseem T. Qazi, Richmond Hill (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/544,530

(22) Filed: Oct. 6, 2006

(51) Int. Cl. A61K 36/42; A61K 36/28; A61K 31/70

(52) U.S. Cl. 424/758; 424/764; 424/776; 514/23; 514/54; 514/560; 514/171; 514/566; 514/769

(57) ABSTRACT

Composition, non-aerosol dispenser containing the composition, and method of raising the blood glucose level of an individual comprising administering an effective amount of the composition to an oral mucosa of the individual. The composition comprises:
a. an effective amount of glucose,
b. an effective amount of at least one absorption enhancer chosen from lecithin, hyaluronic acid, glycolic acid, lactic acid, chamomile extract, cucumber extract, oleic acid, linoleic acid, linolenic acid, monoolein, monooleates, monolaurates, borage oil, evening primrose oil, menthol, trihydroxy oxo cholanyl glycine, glycerin, polyglycerin, lysine, polylysine, triolein, polyoxyethylene ethers, polidocanol alkyl ethers, chenodeoxycholate, deoxycholate, alkali metal salicylate, pharmaceutically acceptable edetate, and pharmaceutically acceptable salts and analogues thereof, and
c. an effective amount of a pharmaceutically acceptable carrier,
wherein the composition is free of any active pharmaceutical agents.

### FIELD OF THE INVENTION

The present invention relates to compositions and methods for raising blood glucose levels, as well as devices containing, and uses of, the compositions.

### BACKGROUND INFORMATION

Hypoglycemia, or low blood glucose (sugar), is a medical condition affecting humans and other mammals. Hypoglycemia exists when blood glucose levels are, or become, lower than normal (typically below 70 mg/dL, and sometimes below 50 mg/dL). Hypoglycemia may occur when plasma insulin levels rise to levels that are higher than normal resulting in blood glucose levels dropping to levels that are insufficient to provide enough energy for normal bodily functions. Hypoglycemia can be exacerbated by exercise or other activities which require higher levels of blood glucose. Hypoglycemia is a well studied side effect of medications used in diabetes treatment, but can also be a side-effect of other medications, or result from other diseases, hormone and enzyme deficiencies, and tumors. Hypoglycemia can also be brought on by exacerbating circumstances in an otherwise healthy individual.

Mild to moderate hypoglycemia can result in symptoms that include one or more of hunger, nervousness, shakiness, perspiration, light-headedness, dizziness, sleepiness, anxiety, weakness, difficulty in speaking or concentrating, nightmares, irritability, slurred speech, confusion, irrational activity, headache, pale skin colour, clumsiness or jerkiness, a tingling sensation around the mouth, tiredness and even seizure. If mild or moderate hypoglycemia is not treated promptly, it can develop into severe hypoglycemia. Severe hypoglycemia occurs when blood glucose levels decrease and remain at a low level for too long. Severe hypoglycemia can cause the brain to become deprived of sufficient energy to function properly, which can result in a loss of consciousness, seizure, coma or death. Other consequences include slurred speech, confusion, and irrational activity.

Hypoglycemia is also fairly common in newborn babies. Symptoms can include jitteriness, cyanosis, apnea, hypothermia, poor body tone, poor feeding, lethargy, and seizures.

The most common reasons for hypoglycemia include inadequate, delayed or skipped meals or snacks, excessive doses of insulin or other diabetes medication such as sulfonylureas and meglitinides, increased activity or exercise, and excessive alcohol consumption.

Individuals with repeated or recurring hypoglycemia may adapt to it, not presenting symptoms of mild to moderate hypoglycemia. This adaptation is especially dangerous, since these individuals can go from being alert and not showing any signs of hypoglycemia directly to a comatose state. The common treatment for such individuals Is frequent blood testing to determine blood glucose levels, and raising blood glucose levels when they become too low. Hypoglycemia can usually be treated relatively quickly and simply, by bringing an individual's blood glucose levels back to normal.

Treatments for hypoglycemia are well established. A common technique is to ingest some form of sugar, such as fruit juice, glucose tablets, milk, sugar, honey, or hard candy, followed by waiting for 15 minutes to see if symptoms abate or blood glucose levels rise to an acceptable level. If symptoms persist or blood glucose levels continue to be below acceptable levels, the individual repeats Ingestion of the sugar containing substance and waits again to see the effect on blood sugar level.

This common technique is only effective if time is not an issue and the affected individual is co-operative and can chew and swallow. Accordingly, there remains a need for therapeutic compositions and administration methods which can be employed conveniently by third parties or self-administered with little or no training.

### SUMMARY OF THE INVENTION

In accordance with a first aspect, the invention provides a glucose composition for delivering glucose across oral mucosa to raise the blood glucose level of an individual, the composition comprising:

an effective amount of glucose,

an effective amount of at least one absorption enhancer chosen from lecithin, hyaluronic acid, glycolic acid, lactic acid, chamomile extract, cucumber extract, oleic acid, linoleic acid, linolenic acid, monoolein, monooleates, monolaurates, borage oil, evening primrose oil, menthol, trihydroxy oxo cholanyl glycine, glycerin, polyglycerin, lysine, polylysine, triolein, polyoxyethylene ethers, polidocanol alkyl ethers, chenodeoxycholate, deoxycholate, alkali metal salicylate, pharmaceutically acceptable edetate, and pharmaceutically acceptable salts and analogues thereof, and

an effective amount of a pharmaceutically acceptable carrier,

wherein the composition is free of any additional active pharmaceutical agents.

The oral mucosa may be the buccal mucosa.

The composition may contain glucose in a concentration of from 10 to 80 w/w %, 20 to 70 w/w %, or from 30 to 60 w/w %.

The absorption enhancer may be present in a concentration of from 0.01 to 5 w/w %, 0.01 to 3 w/w %, or from 0.01 to 1 w/w %. The total concentration of absorption enhancer may be less than 10 w/w %, or less than 5 w/w %.

The composition may comprise sodium glycocholate, glycerin, sodium lauryl sulfate, and/or polyoxyethylene sorbitan monooleate.

The carrier may be an aqueous solvent such as purified water. Alternatively, the carrier may be a chewing gum base.

The composition may further comprise at least one additional ingredient chosen from flavouring agents, colouring agents, preservatives and antimicrobial agents. Furthermore, the composition may comprise sodium benzoate as a preservative or antimicrobial agent.

In accordance with a second aspect, the invention provides a non-aerosol dispenser containing a composition according to the first aspect. The dispenser may be a metered dose dispenser which may be adapted to deliver the composition as droplets having a size between 1 and 200 μm or 1 and 20 μm, and may be adapted to dispense between 0.05 and 0.5 mL, 0.05 and 0.3 mL, or 0.05 and 0.1 mL of the glucose composition per actuation.

In accordance with a third aspect, the invention provides a method of raising the blood glucose level of an individual comprising administering an effective amount of the composition according to the first aspect to an oral (e.g. buccal) mucosa of the individual. The administration may comprise spraying the composition into the mouth of the individual using a non-aerosol dispenser (e.g. a metered dose dispenser).

The method may comprise administering 30 to 50 mg of glucose per actuation of the dispenser, and may comprise administering a total dose of from 150 to 300 mg or 175 to 200 mg glucose in one dosing session. Furthermore, the method may comprise administering three to eight spray actuations per dosing session.

The amount of glucose composition sprayed per actuation may be between 0.05 and 0.5 mL, 0.05 and 0.3 mL, or 0.05 and 0.1 mL.

In accordance with a fourth aspect, the invention provides a use of the composition according to the first aspect in raising the blood glucose level of an individual, or in treating hypoglycemia in an individual.

Finally, in accordance with a fifth aspect, the invention provides a use of the composition according to the first aspect In the manufacture of a product for treating hypoglycemia or low blood sugar.

The present invention provides a glucose composition in which glucose is, at least in part, encapsulated in micelles formed by one or more of the disclosed absorption enhancing agents or enhancers. The glucose may be present in the form of small unilamellar vesicles, large unilamellar vesicles, multilamellar vesicles, or oligolamellar vesicles. The absorption enhancer allows glucose to be rapidly transported across oral membranes to increase blood glucose levels in individuals in need of treatment. The oral membranes or mucosae consist of the buccal, pharyngeal, lingual, sublingual and palate mucosae. The sublingual mucosa includes the membrane of the ventral surface of the tongue and the floor of the mouth, and the buccal mucosa is the lining of the cheeks. The sublingual, buccal and other oral mucosae are relatively permeable, allowing for rapid absorption of the present composition. Consequently, the present method of raising the blood glucose level of an individual is effective, non-invasive and convenient.

### BRIEF DESCRIPTION OF THE DRAWINGS

FIG. 1 shows the effect of oral administration of a glucose composition according to the invention on blood glucose level in non-fasting individuals with Type II diabetes.

FIG. 2 shows the effect of oral administration of the glucose composition on blood glucose level in fasting individuals with Type II diabetes.

FIG. 3 shows the effect of oral administration of the glucose composition on blood glucose level in non-fasting, healthy individuals.

FIG. 4 shows the effect of oral administration of the glucose composition on blood glucose level in fasting, healthy individuals.

FIG. 5 shows the effects of oral administration of the glucose composition containing 4 grams of glucose on blood glucose level in fasting, healthy Individuals to whom were administered insulin in an amount of 0.11 IU/kg body weight. A placebo curve and a curve representing ingestion of a glucose tablet containing 4 grams of glucose are provided for comparison purposes.

FIG. 6 shows the effect of oral administration of the glucose composition containing 8 grams of glucose on blood glucose level in fasting, healthy individuals. A placebo curve and a curve representing ingestion of two glucose tablets containing 8 grams of glucose are provided for comparison purposes.

FIG. 7 shows the effect of oral administration of the glucose composition containing 4 grams of glucose on blood glucose level in fasting, healthy individuals. A placebo curve and a curve representing ingestion of a glucose tablet containing 4 grams of glucose are provided for comparison purposes.

### DETAILED DESCRIPTION OF THE INVENTION

The term “comprising”, when used herein, means “including without limitation”. Thus, a formulation or group comprising a number of integers may also comprise additional integers not specifically recited. The term “consisting essentially of” when used herein means including the recited Integers and such additional integers that do not materially affect the basic and novel properties of the invention. The basic and novel properties of the invention are the absorption characteristics of the glucose contained in the present composition through the oral mucosae (e.g. buccal, pharyngeal, lingual, sublingual and palate mucosae) into a individual's bloodstream. The term “consisting of”, when used herein, means “including only the recited ingredients and any impurities normally contained in same”. Thus, a formulation or group consisting of a number of integers may not comprise additional integers not specifically recited apart from the impurities normally contained in the recited integers.

As used herein, the term “effective amount” refers to that amount needed to bring about the desired result, i.e. obtaining the intended rise in blood glucose level in an individual at the desired rate of absorption of glucose across oral membranes. Such an amount will be understood as having a therapeutic effect in an individual.

It will be appreciated that the effective amount will vary depending on the glucose composition, the nature and severity of the hypoglycemia being treated, and the individual being treated. The determination of what constitutes an “effective amount” for a particular individual is within the skill of one practicing in the art based upon the general guidelines provided herein.

All expressions of concentration (i.e. w/w %) used in the present specification, including in the claims, are in terms of the total weight of the composition, unless otherwise specified.

As used herein, the terms “individual” and “individuals” refer to members of the animal kingdom, including but not limited to humans.

When used herein, the term “active pharmaceutical agent” means an ingredient useful in treating or preventing an illness, disease or abnormal state in a human or animal body and includes glucose for the treatment of hypoglycemia.

The pharyngeal, sublingual, lingual, palate and buccal mucosa are collectively referred to herein as the “oral mucosae”. “Oral mucosa” means any one of the pharyngeal, sublingual, lingual, palate and buccal mucosa.

### Glucose Composition

According to one embodiment, the glucose composition is present in liquid form. The concentration of the glucose contained in the composition can range from 100-800 g of glucose per litre, preferably between 300-800 g of glucose per litre, and more preferably about 500 g of glucose per litre. Alternatively, the glucose may be present in a concentration of from 10 to 80 w/w %, from 20 to 70 w/w %, or from 30 to 60 w/w %.

The present composition contains at least one absorption enhancer to enhance the transport of glucose across oral membranes. The enhancer serves to encapsulate the glucose in micelles. Mixed micelles are formed when more than one absorption enhancer is used. Each absorption enhancer is present in a concentration of between 0.01 and 5, preferably between 0.01 and 2, and more preferably between 0.01 and 1 wt./wt. %. The total amount of absorption enhancers is less than 10, preferably less than 5, and more preferably less than 2 wt./wt. %.

Any alkali metal alkyl sulfate can be used in the present compositions provided compatibility problems do not arise. Preferably, the alkyl is a C8 to C22 alkyl, more preferably lauryl (C12). Any alkali metal can be utilized, with sodium being preferred. If present, the concentration of the alkali metal alkyl sulfate is preferably between 0.01 and 3, 0.01 and 2, and 0.01 and 1 wt./wt.%.

It will be appreciated that several of the absorption enhancers are generally described as fatty acids, bile acids, or salts thereof. A particularly preferred absorption enhancer is trihydroxy cholanyl glycine and pharmaceutically acceptable salts thereof (e.g. sodium glycocholate). When used, sodium glycocholate is preferably present in a concentration of between 0.01 and 5, 0.01 and 3, and 0.01 and 1 wt./wt.%.

Glycerin is another preferred absorption enhancer. When present, it exists in a concentration of between 0.01 and 15, 0.01 and 10, 0.01 and 5, and 0.01 and 2 wt./wt.%.

The lecithin can be saturated or unsaturated, and is preferably selected from the group consisting of phosphatidylcholine, phosphatidylserine, sphingomyelin, phosphatidylethanolamine, cephalin, and lysolecithin.

Preferred salts of hyaluronic acid are alkali metal hyaluronates, especially sodium hyaluronate, alkaline earth hyaluronates, and aluminum hyaluronate.

Particularly suitable absorption enhancers include i) glycerin, sodium glycocholate, and sodium lauryl sulfate, and ii) glycerin, sodium glycocholate, and polyoxyethylene sorbitan monooleate (sold in association with the trademark Tween 80).

The present compositions optionally contain a stabilizer, preservative, and/or antioxidant. Phenolic compounds are particularly suited for this purpose as they not only stabilize the compositions, but they also protect against bacterial growth and help with absorption of the composition. A phenolic compound will be understood as referring to a compound having one or more hydroxy groups attached directly to a benzene ring. Preferred phenolic compounds according to the present invention include phenol and methyl phenol (also known as m-cresol), and mixtures thereof. Known sugar preservatives, such as sodium benzoate, can also be used advantageously. Antioxidants include ascorbal palmitate, BHA and BHT.

It will be understood by those skilled in the art that colourants, flavouring agents, taste masking agents, and non-therapeutic amounts of other compounds may also be included in the formulation. Typical flavouring and taste masking agents include peppermint oil, menthol, synthetic flavours of strawberry, orange, cherry, pomegranate, raspberry, grape, mango, banana, watermelon, chocolate, vanilla, and other flavours. When menthol is used as one of the absorption enhancing agents, obviously, it will also impart flavour to the composition. A typical colourant which may be used is a food-grade colour (e.g. red food colour).

An isotonic agent such as glycerin or dibasic sodium phosphate may also be added to the composition. The isotonic agent serves to keep the micelles in composition. When glycerin is used as one of the absorption enhancing agents it will also function as an isotonic agent. When dibasic sodium phosphate is used as an isotonic agent, it will also serve to inhibit bacterial growth.

The pH of the present glucose composition should typically be in the range of 3.5 to 7. Hydrochloric acid, sodium hydroxide or another known base or acid, can be utilized to adjust the pH of the composition if needed.

The compositions of the present invention may be stored at temperatures ranging from 15° C. to 40° C., and preferably from 15° C. to 30° C.

The present glucose composition is preferably in liquid form for spray administration. However, it can also be manufactured in other dosage forms by changing, amongst other things, the pharmaceutically acceptable carrier that is employed. For example, the present composition can be made into chewing gum, chewing gum with a liquid centre, chewable tablets, and lozenges. One skilled in the art of pharmaceutical formulation would be able to formulate a composition in these other dosage forms. It will be appreciated that the glucose, contained in the chewing gum, chewing gum with liquid centre, chewable tablets or lozenges, is administered across oral membranes (predominantly) rather than through membranes of the gastro-intestinal tract. For this to occur, the composition is held in the mouth for a certain minimum amount of time which can be determined readily through simple experimentation.

### Process for Making Glucose Composition

The present invention also provides a method for making the glucose composition of the present invention. The present compositions may be prepared by mixing an aqueous solution of glucose with at least one absorption enhancer and other additives or ingredients. The glucose should be added in an amount effective for the desired purpose. The absorption enhancers may be added concurrently or sequentially. Micelles will form with substantially any kind of mixing of the ingredients but vigorous mixing in a high speed, high shear homogenizing mixer is preferred. The glucose, absorption enhancers, and other ingredients described above are all suitable for use in the present methods.

According to one method, the composition is prepared by making a glucose solution, making an “excipient” solution and mixing the two solutions together. The excipient solution is prepared by adding one or more absorption enhancers and any other ingredients (e.g. preservative, flavouring agent, colouring agent, etc.) to an aqueous solvent (e.g. purified water).

### Devices for Administration

Liquid embodiments of glucose compositions according to the invention can be administered using a variety of non-aerosol mechanical pump spray devices that are known in the art. Such devices can administer the composition in the form of an atomized spray or mist. A benefit of many such devices is that the potential for contamination is minimized because the devices are self-contained.

The non-aerosol dispenser may contain a metering pump pre-set to deliver a pre-defined amount of the glucose composition, or it may be adjustable by the user to deliver varying amounts. One benefit of using a device having a metering pump (e.g. metered dose dispenser) is the ability to deliver a precise amount of composition with each actuation. Preferably, the non-aerosol metered dose dispenser is pre-set to deliver an amount that is less than (i.e. a fraction of) the recommended dose, so that the individual may have greater control over the amount of the glucose composition that is administered in one session. For example, the dispenser can be set such that each administration or actuation of the dispenser yields about 50 mg of glucose. This way, individuals can adjust the amount administered in 50 mg intervals. For example, an individual can administer 3 actuations for a low dose, or 5 actuations for a higher dose.

The devices can vary in size, both in terms of the container capacity and in terms of the size of the pump, and hence quantity of the composition administered per actuation and per session. Container sizes can have capacities varying from 5 to 100 mL, or from 10 to 50 mL, or from 20 to 30 mL. Also, the metering pump can be configured to deliver from 0.05 to 0.5 mL, or from 005 to 0.3 mL, or from 0.05 to 0.1 mL per actuation.

### Method of Administration

The glucose composition according to the invention (in liquid form) can be administered quickly and easily by the subject individual or by a third party with little or no training (if the individual is unconscious or uncooperative until emergency assistance arrives). Self-administration can be done “to effect”, i.e. the individual can continue administration until the individual feels the symptoms disappear. Alternatively, the administration can be of a defined quantity of the glucose composition (e.g. using a metered dose dispenser) followed by a waiting period to determine whether the symptoms disappear. Administration is by spraying the glucose composition into the mouth with a non-aerosol dispenser. Preferably, the composition is sprayed into the oral cavity without inhalation, so that the droplets stay in the mouth rather than being drawn into the lungs, and the glucose is absorbed across oral membranes or mucosae.

In accordance with another method, if the individual is conscious, and where the composition is in the form of a chewing gum (with or without a liquid centre) or tablet, the chewing gum or tablet is chewed and held in the mouth until the desired rise in blood glucose level is achieved, as experienced by reduction or elimination of symptoms of low blood sugar.

One embodiment of the present inventive composition was prepared at follows:

### EXAMPLE 1

### Preparation of a Glucose Composition According to Present Invention

### Solution A:

350 mL purified water USP was heated to approximately 90° C. 500 g of glucose was added. The water/glucose solution was stirred until all of the glucose was dissolved. The water/glucose solution was then allowed to return to approximately room temperature, while stirring.

### Solution B:

While stirring, the following were added to 100 mL purified water USP: 11 g glycerin, 2 g sodium glycocholate, 500 mg of sodium lauryl sulfate, 500 mg sodium benzoate, 2 g orange flavour, 2 grams of artificial cooling flavour (such as that sold by a Swiss company, Givaudan). Each ingredient was added, in turn, and made to dissolve before the next ingredient was added.

Solutions A and B were then combined and purified water USP was added to a total volume of 1 L. The resultant glucose composition was then stirred for about 5 minutes and stored at a temperature between 15° C. to 30° C.

The above glucose composition is summarized in the below table:

Glucose Composition

| Ingredient | Amount (g/mL) | | Amount (w/w %) |
|---|---|---|---|
| Glucose | 0.5000 | | 42.37 |
| Glycerin | 0.0110 | | 0.93 |
| Sodium glycocholate | 0.0020 | | 0.17 |
| Sodium lauryl sulfate | 0.0005 | | 0.04 |
| Sodium benzoate | 0.0005 | | 0.04 |
| Orange flavour | 0.0020 | | 0.17 |
| Cooling flavour | 0.0020 | | 0.17 |
| Purified water USP | QS to 1 | mL | QS to 100 |

### EXAMPLE 2

### Use of Glucose Composition in a Non-Aerosol Metered Dose Dispenser

5 mL of the glucose composition summarized in the above table in Example 1 was loaded into a non-aerosol pump dispenser equipped with a dip tube.

The glucose composition was sprayed out of the dispenser numerous times, and the mass (and resultant volume) of glucose composition sprayed per actuation was measured. The average weight was found to be 0.084 g of the glucose composition, with a minimum weight of 0.077 g, and a maximum weight of 0.088 g.

The density of the glucose composition was calculated to be 1.1532 g/ml. Thus, the average volume of the composition dispensed per actuation was found to be 0.073 ml, with a minimum volume of 0.067 ml, and a maximum volume of 0.076 ml.

Based on the amount of glucose in the glucose composition (500 g/L), the average quantity of glucose per actuation was calculated to be 0.036 g, with a minimum glucose quantity of 0.033 g, and a maximum glucose quantity of 0.038 grams. Actuating the dispenser five times would therefore cause 0.180 g of glucose to be administered in total, on average, with a minimum total of about 0.165 g and a maximum total of about 0.190 g.

The present method and composition produce a rise in blood glucose levels as illustrated by the below examples.

### EXAMPLE 3

### Effect of Administration of Glucose Composition on Non-Fasting Individual with Type II Diabetes

The glucose composition of Example 1 was administered using the metered dose dispenser of Example 2 to a non-fasting individual with Type II diabetes and blood glucose level was monitored.

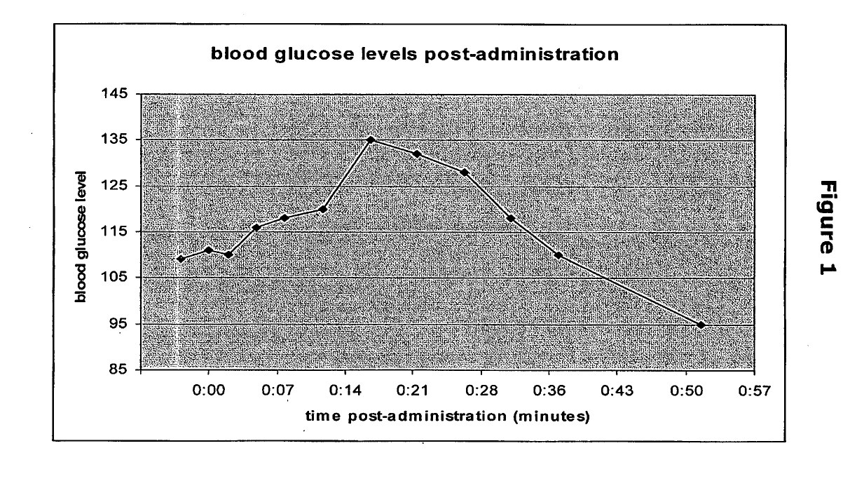

The individual self-administered the composition to the buccal cavity by actuating the dispenser five times, without inhaling or swallowing. Blood glucose level was measured before, during, and after administration using standard methods. The glucose composition was administered at time shortly before “0”. The results are listed in Table 1 and plotted in FIG. 1. The measurements show a rise in blood glucose levels in under 5 minutes, and a peak plasma glucose concentration being reached in about 17 minutes.

TABLE 1
Effect of administration of glucose composition on non-
fasting individual with Type II diabetes

| Minutes post-administration | Blood glucose level (mg/dl) |
|---|---|
| −3 | 109 |
| 0* | 111 |
| 2 | 110 |
| 5 | 116 |
| 8 | 118 |
| 12 | 120 |
| 17 | 135 |
| 22 | 132 |
| 27 | 128 |
| 32 | 118 |
| 37 | 110 |
| 52 | 95 |

*measured as soon as possible after composition was administered.

### EXAMPLE 4

### Effect of Administration of Glucose Composition on Fasting Individual with Type II Diabetes

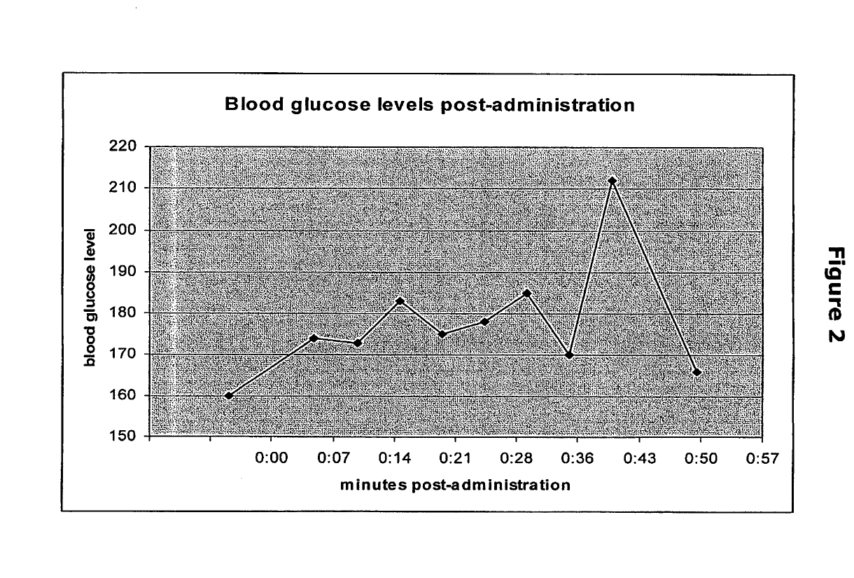

The glucose composition of Example 1 was administered using the metered dose dispenser of Example 2 to a fasting individual with Type II diabetes and blood glucose level was monitored. After fasting for 12 hours, the individual self-administered the composition to the buccal cavity by actuating the dispenser five times, without inhaling or swallowing. Blood glucose level was measured before, during, and after administration using standard methods. The glucose composition was administered at time shortly before “0” The results are listed in Table 2 and plotted in FIG. 2. The measurements show an almost instantaneous rise in blood glucose level, and a peak plasma glucose concentration being reached in about 40 minutes.

TABLE 2
Effect of administration of glucose composition on fasting
individual with Type II diabetes

| Minutes post-administration | Blood glucose level (mg/dl) |
|---|---|
| −5 | 160 |
| 0* | 174 |
| 10 | 173 |
| 15 | 183 |
| 20 | 175 |
| 25 | 178 |
| 30 | 185 |
| 35 | 170 |
| 40 | 212 |
| 50 | 166 |

*measured as soon as possible after composition was administered.

### EXAMPLE 5

### Effect of Administration of Glucose Composition on Healthy, Non-Fasting Individual

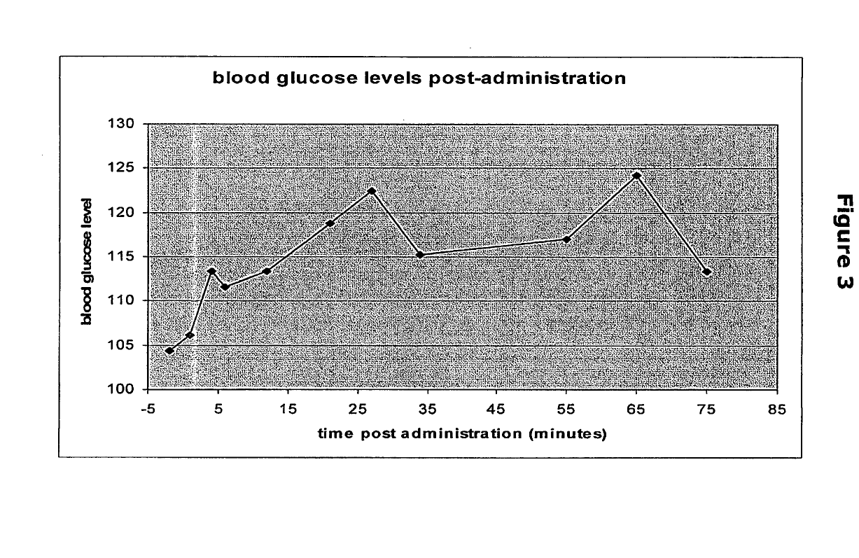

The glucose composition of Example 1 was administered using the metered dose dispenser of Example 2 to a healthy non-fasting individual and blood glucose level was monitored. The Individual self-administered the composition to the buccal cavity by actuating the dispenser five times, without inhaling or swallowing. Blood glucose level was measured before, during, and after administration using standard methods. The glucose composition was administered at time shortly before “0”. The results are listed in Table 3 and plotted in FIG. 3. The measurements show a rise in blood glucose levels in under 4 minutes, a first peak plasma glucose concentration being reached at about 27 minutes and a second slightly higher peak plasma glucose concentration being reached at about 65 minutes.

TABLE 3
Effect of glucose composition on non-fasting healthy individual

| Minutes post administration | Blood glucose level (mg/dl) |
|---|---|
| −2 | 104.4 |
| 1 | 106.2 |
| 4 | 113.4 |
| 6 | 111.6 |
| 12 | 113.4 |
| 21 | 118.8 |
| 27 | 122.4 |
| 34 | 115.2 |
| 55 | 117 |
| 65 | 124.2 |
| 75 | 113.4 |

### EXAMPLE 6

### Effect of Administration of Glucose Composition on Healthy, Fasting Individual

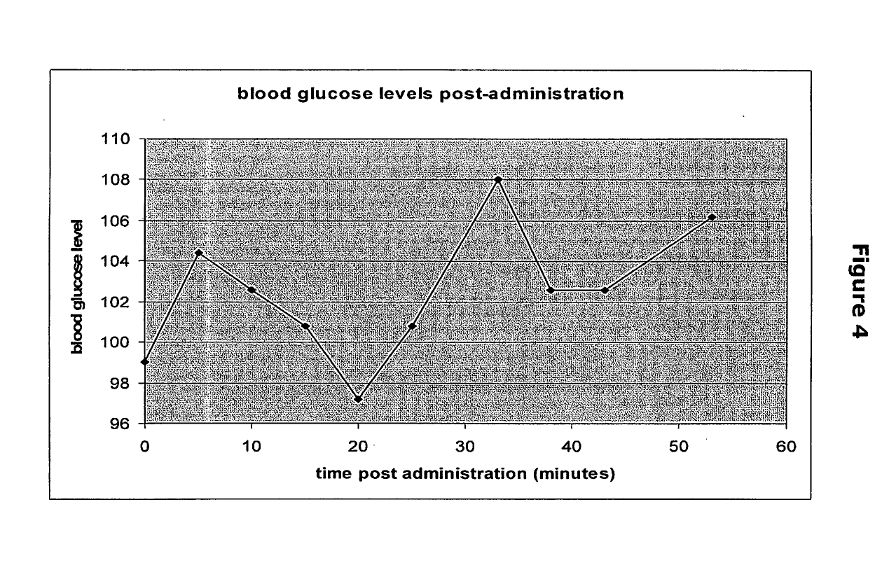

The glucose composition of Example 1 was administered using the metered dose dispenser of Example 2 to a fasting, healthy individual and blood glucose level was monitored. After fasting for 12 hours, the individual self-administered the composition to the buccal cavity by actuating the dispenser five times, without inhaling or swallowing. Blood glucose level was measured before, during, and after administration using standard methods. The glucose composition was administered at time shortly before “0”. The results are listed in Table 4 and plotted in FIG. 4. The measurements show a rise in blood glucose level in under 5 minutes, a first peak plasma glucose concentration being reached in about 33 minutes and a second slightly higher peak plasma glucose concentration being reached in about 53 minutes.

TABLE 4
Effect of administration of glucose composition on fasting
healthy individual

| Minutes post-administration | Blood glucose level (mg/dl) |
|---|---|
| 0* | 99 |
| 5 | 104.4 |
| 10 | 102.6 |
| 15 | 100.8 |
| 20 | 97.2 |
| 25 | 100.8 |
| 33 | 108 |
| 38 | 102.6 |
| 43 | 102.6 |
| 53 | 106.2 |

*measured as soon as possible after composition was administered.

In the following Examples 7 to 9, all subjects fasted from 8 pm the night before until the tests were done the following morning at 7 am.

### EXAMPLE 7

### Glucodynamics After Insulin Induced Hypoglycemia (0.11 IU/kg Weight of S.C. Regular Insulin) and Administration of 4 Grams Glucose Composition and 4 Grams Tablet at 70 Minutes

11 subjects were given 0.11 IU/kg body weight insulin by subcutaneous injection at time=0 minutes. At time equals 70 minutes, the subjects were given a commercial glucose tablet (Dex®4 Glucose Tablets, Orange, manufactured by AMG Medical Inc., Montreal, Quebec) containing 4 grams of glucose. The tablet was chewed and swallowed. Blood glucose levels (mg/dL) of the subjects were measured at intervals from time=−15 minutes.

On a second day, the same 11 subjects were given 0.11 IU/kg body weight insulin at time=0 minutes. At time equals 70 minutes, the subjects were given the glucose composition described above according to the present invention. The glucose composition was sprayed into the oral cavity using a standard non-aerosol dispenser equipped with a dip tube and glucose was absorbed through the oral membranes and residual liquid swallowed. The amount of the glucose composition administered and swallowed was equivalent to 4 grams of glucose. Blood glucose levels (mg/dL) of the subjects were measured at intervals from time=−15 minutes.

To provide a placebo comparison, on a third day, 12 subjects, seven of whom participated in the above trials, were not given insulin, glucose tablets or the present glucose composition. Their blood glucose levels were simply measured at regular intervals starting at time=−10 minutes.

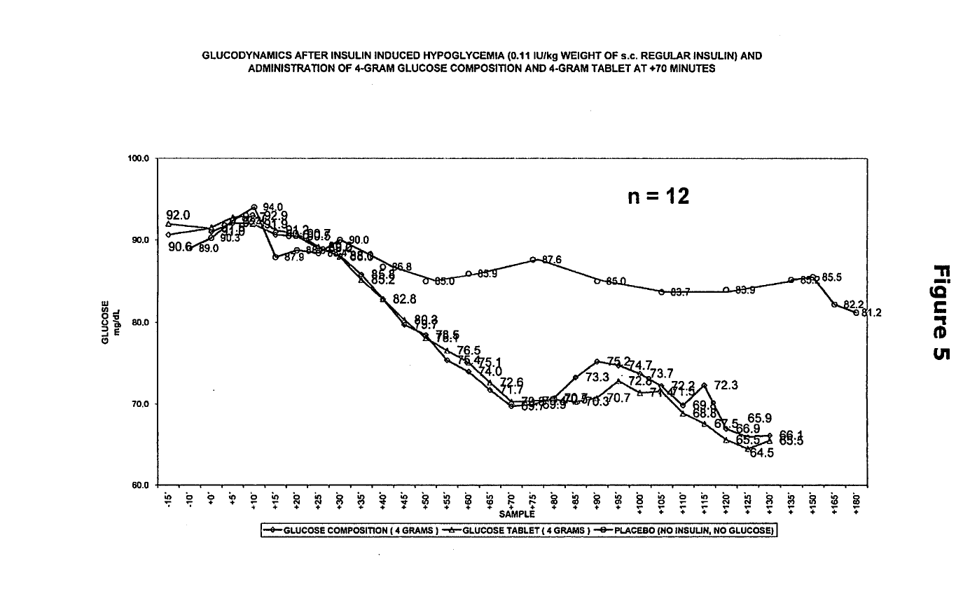

The mean blood glucose level values are summarized below and plotted in FIG. 5.

GLUCODYNAMICS AFTER INSULIN INDUCED HYPOGLYCEMIA (0.11 IU/kg
WEIGHT OF S.C. REGULAR INSULIN) AND ADMINISTRATION OF
4-GRAM GLUCOSE COMPOSITION AND 4-GRAM TABLET AT +70 MINUTES
Mean

| | 1 | 2 | 3 | 4 | 5 | 6 | 7 |
|---|---|---|---|---|---|---|---|
| | −15′ | +0′ | +5′ | +10′ | +15′ | +20′ | +25′ |
| Glucose Composition | 90.6 | 91.0 | 92.1 | 91.9 | 90.6 | 90.5 | 89.0 |
| (4 grams) | | | | | | | |
| SD | 4.8 | 5.3 | 5.3 | 6.7 | 5.3 | 6.6 | 6.4 |
| | 8 | 9 | 10 | 11 | 12 | 13 | 14 |
| | +30′ | +35′ | +40 ′ | +45′ | +50′ | +55′ | +60′ |
| Glucose Composition | 88.1 | 85.8 | 82.8 | 79.7 | 78.5 | 75.4 | 74.0 |
| (4 grams) | | | | | | | |
| SD | 7.6 | 7.4 | 7.9 | 9.2 | 10.9 | 11.5 | 12.2 |
| | 15 | 16 | 17 | 18 | 19 | 20 | 21 |
| | +65′ | +70′ | +75′ | +80′ | +85′ | +90′ | +95′ |
| Glucose Composition | 71.7 | 69.7 | 69.9 | 70.7 | 73.3 | 75.2 | 74.7 |
| (4 grams) | | | | | | | |
| SD | 14.2 | 14.4 | 14.9 | 13.8 | 14.5 | 13.6 | 15.5 |
| | 22 | 23 | 24 | 25 | 26 | 27 | 28 |
| | +100′ | +105′ | +110′ | +115′ | +120′ | +125′ | +130′ |
| Glucose Composition | 73.7 | 72.2 | 69.8 | 72.3 | 66.9 | 65.9 | 66.1 |
| (4 grams) | | | | | | | |
| SD | 13.0 | 13.4 | 12.6 | 19.1 | 11.4 | 11.9 | 10.3 |
| | 1 | 2 | 3 | 4 | 5 | 6 | 7 |
| | −15′ | +0′ | +5′ | +10′ | +15′ | +20′ | +25′ |
| GLUCOSE TABLET | 92.0 | 91.5 | 92.7 | 92.9 | 91.2 | 90.7 | 89.2 |
| (4 grams) | | | | | | | |
| SD | 3.7 | 4.3 | 3.6 | 4.5 | 5.0 | 3.9 | 4.0 |
| | 8 | 9 | 10 | 11 | 12 | 13 | 14 |
| | +30′ | +35′ | +40′ | +45′ | +50′ | +55′ | +60′ |
| GLUCOSE TABLET | 88.0 | 85.2 | 82.8 | 80.3 | 78.1 | 76.5 | 75.1 |
| (4 grams) | | | | | | | |
| SD | 3.3 | 3.5 | 4.0 | 4.8 | 5.3 | 6.7 | 7.3 |
| | 15 | 16 | 17 | 18 | 19 | 20 | 21 |
| | +65′ | +70′ | +75′ | +80′ | +85′ | +90′ | +95′ |
| GLUCOSE TABLET | 72.6 | 70.3 | 70.4 | 70.5 | 70.3 | 70.7 | 72.8 |
| (4 grams) | | | | | | | |
| SD | 7.6 | 9.0 | 8.9 | 7.6 | 8.4 | 10.4 | 12.2 |
| | 22 | 23 | 24 | 25 | 26 | 27 | 28 |
| | +100′ | +105′ | +110′ | +115′ | +120′ | +125′ | +130′ |
| GLUCOSE TABLET | 71.4 | 71.5 | 68.8 | 67.5 | 65.5 | 64.5 | 65.5 |
| (4 grams) | | | | | | | |
| SD | 11.4 | 10.1 | 9.9 | 9.5 | 9.1 | 9.1 | 8.6 |

| | 1 | 2 | 3 | 4 | 5 | 6 | 7 | 8 | 9 |
|---|---|---|---|---|---|---|---|---|---|
| | −10′ | +0′ | +5′ | +10′ | +15′ | +20′ | +25′ | +30′ | +40′ |
| PLACEBO | 89.00 | 90.25 | 92.33 | 94.00 | 87.92 | 88.75 | 88.42 | 90.00 | 86.75 |
| SD | 4.8 | 4.0 | 3.0 | 5.8 | 4.0 | 4.4 | 4.8 | 5.2 | 5.4 |

| | 10 | 11 | 12 | 13 | 14 | 15 | 16 | 17 | 18 | 19 |
|---|---|---|---|---|---|---|---|---|---|---|
| | +50′ | +60′ | +75′ | +90′ | +105′ | +120′ | +135′ | +150′ | +165′ | +180′ |
| PLACEBO | 85.00 | 85.92 | 87.58 | 85.00 | 83.67 | 83.92 | 85.17 | 85.50 | 82.17 | 81.17 |
| SD | 6.3 | 6.8 | 7.2 | 7.0 | 5.9 | 6.6 | 6.8 | 5.9 | 7.1 | 6.2 |

SD = standard deviation

FIG. 5 shows the present inventive glucose composition as comparable to or outperforming glucose tablets under these test conditions.

### EXAMPLE 8

### Comparison Between 8 Grams Glucose Tablets Vs. 8 Grams Glucose Composition vs. Placebo

12 subjects were given the glucose composition described above according to the present invention. The glucose composition was sprayed into the oral cavity using a standard non-aerosol dispenser equipped with a dip tube and glucose was absorbed through the oral membranes and residual liquid swallowed. The amount of the glucose composition administered and swallowed was equivalent to 8 grams of glucose. Blood glucose levels (mg/dL) of the subjects were measured at intervals from time=−10 minutes.

On a second day, 12 subjects were given two commercial glucose tablets (Dex®4 Glucose Tablets, Orange, manufactured by AMG Medical Inc., Montreal, Quebec) each containing 4 grams of glucose. The tablets were chewed and swallowed. Blood glucose levels (mg/dL) of the subjects were measured at intervals from time=−10 minutes.

To provide a placebo comparison, on a third day, 12 subjects were not given glucose tablets or the present glucose composition. Their blood glucose levels were simply measured at regular intervals starting at time=−10 minutes.

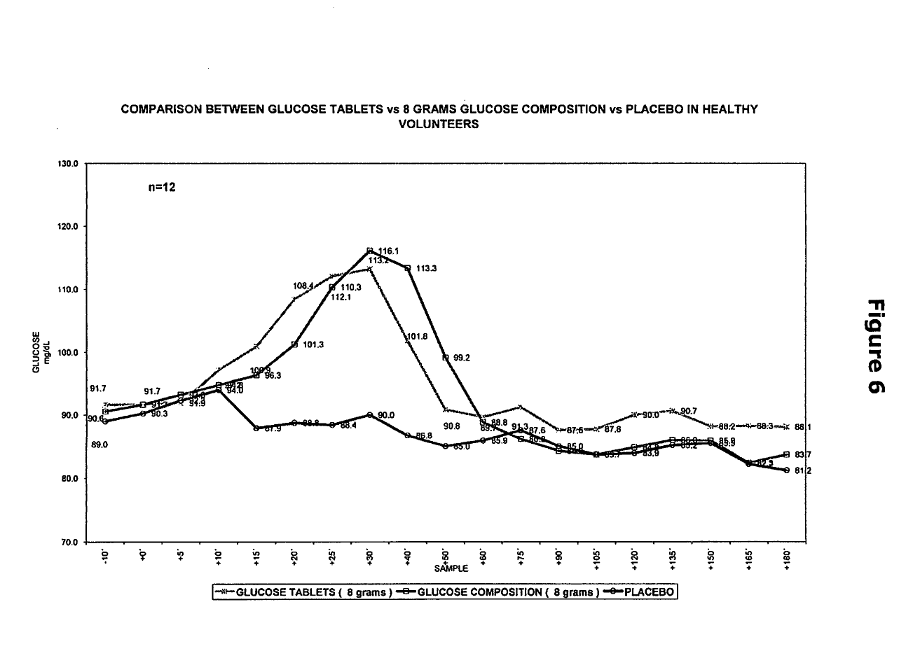

The mean blood glucose level values are summarized below and plotted in FIG. 6.

COMPARISON BETWEEN GLUCOSE TABLETS vs. 8 GRAMS GLUCOSE
COMPOSITION vs. PLACEBO IN HEALTHY VOLUNTEERS
Mean

| | 1 | 2 | 3 | 4 | 5 | 6 | 7 | 8 | 9 |
|---|---|---|---|---|---|---|---|---|---|
| | −10′ | +0′ | +5′ | +10′ | +15′ | +20′ | +25′ | +30′ | +40′ |
| Glucose | 90.6 | 91.7 | 93.3 | 94.8 | 96.3 | 101.3 | 110.3 | 116.1 | 113.3 |
| Composition | | | | | | | | | |
| (8 grams) | | | | | | | | | |
| SD | 4.2 | 5.2 | 5.4 | 7.8 | 8.0 | 8.6 | 10.8 | 10.1 | 13.3 |

| | 10 | 11 | 12 | 13 | 14 | 15 | 16 | 17 | 18 | 19 |
|---|---|---|---|---|---|---|---|---|---|---|
| | +50′ | +60′ | +75′ | +90′ | +105′ | +120′ | +135′ | +150′ | +165′ | +180′ |
| Glucose | 99.2 | 88.8 | 86.2 | 84.3 | 83.7 | 84.8 | 86.0 | 85.9 | 82.3 | 83.7 |
| Composition | | | | | | | | | | |
| (8 grams) | | | | | | | | | | |
| SD | 11.2 | 10.0 | 4.9 | 6.3 | 6.0 | 5.7 | 6.3 | 6.0 | 5.4 | 6.2 |

| | | 1 | 2 | 3 | 4 | 5 | 6 | 7 | 8 | 9 |
|---|---|---|---|---|---|---|---|---|---|---|
| | | −10′ | +0′ | +5′ | +10′ | +15′ | +20′ | +25′ | +30′ | +40′ |
| Glucose Tablets | | 91.7 | 91.7 | 91.9 | 97.2 | 100.9 | 108.4 | 112.1 | 113.2 | 101.8 |
| (8 grams) | | | | | | | | | | |
| SD | | 3.7 | 4.3 | 5.2 | 4.1 | 6.7 | 10.2 | 12.1 | 11.3 | 9.4 |
| | 10 | 11 | 12 | 13 | 14 | 15 | 16 | 17 | 18 | 19 |
| | +50′ | +60′ | +75′ | +90′ | +105′ | +120′ | +135′ | +150′ | +165′ | +180′ |
| Glucose | 90.8 | 89.7 | 91.3 | 87.6 | 87.8 | 90.0 | 90.7 | 88.2 | 88.3 | 88.1 |
| Tablets | | | | | | | | | | |
| (8 grams) | | | | | | | | | | |
| SD | 9.0 | 6.5 | 4.8 | 4.6 | 4.9 | 5.3 | 5.3 | 5.0 | 4.7 | 4.6 |

| | 1 | 2 | 3 | 4 | 5 | 6 | 7 | 8 | 9 |
|---|---|---|---|---|---|---|---|---|---|
| | −10′ | +0′ | +5′ | +10′ | +15′ | +20′ | +25′ | +30 ′ | +40′ |
| PLACEBO | 89.00 | 90.25 | 92.33 | 94.00 | 87.92 | 88.75 | 88.42 | 90.00 | 86.75 |
| SD | 4.8 | 4.0 | 3.0 | 5.8 | 4.0 | 4.4 | 4.8 | 5.2 | 5.4 |

| | 10 | 11 | 12 | 13 | 14 | 15 | 16 | 17 | 18 | 19 |
|---|---|---|---|---|---|---|---|---|---|---|
| | +50′ | +60′ | +75′ | +90′ | +105′ | +120′ | +135′ | +150′ | +165′ | +180′ |
| PLACEBO | 85.00 | 85.92 | 87.58 | 85.00 | 83.67 | 83.92 | 85.17 | 85.50 | 82.17 | 81.17 |
| SD | 6.3 | 6.8 | 7.2 | 7.0 | 5.9 | 6.6 | 6.8 | 5.9 | 7.1 | 6.2 |

SD = standard deviation

FIG. 6 shows that the present inventive glucose composition is comparable to or better than glucose tablets in terms of its induced glucodynamic response.

### EXAMPLE 9

### Comparison Between 4 Grams Glucose Tablets vs. 4 Grams Glucose Composition Vs. Placebo

12 subjects were given the glucose composition described above according to the present invention. The glucose composition was sprayed into the oral cavity using a standard non-aerosol dispenser equipped with a dip tube and glucose was absorbed through the oral membranes and residual liquid swallowed. The amount of the glucose composition administered and swallowed was equivalent to 4 grams of glucose. Blood glucose levels (mg/dL) of the subjects were measured at intervals from time=−10 minutes.

On a second day, 12 subjects were given a commercial glucose tablet (Dex®4 Glucose Tablets, Orange, manufactured by AMG Medical Inc., Montreal, Quebec) containing 4 grams of glucose. The tablet was chewed and swallowed. Blood glucose levels (mg/dL) of the subjects were measured at intervals from time=−10 minutes.

To provide a placebo comparison, on a third day, 12 subjects were not given glucose tablets or the present glucose composition. Their blood glucose levels were simply measured at regular intervals starting at time=−10 minutes.

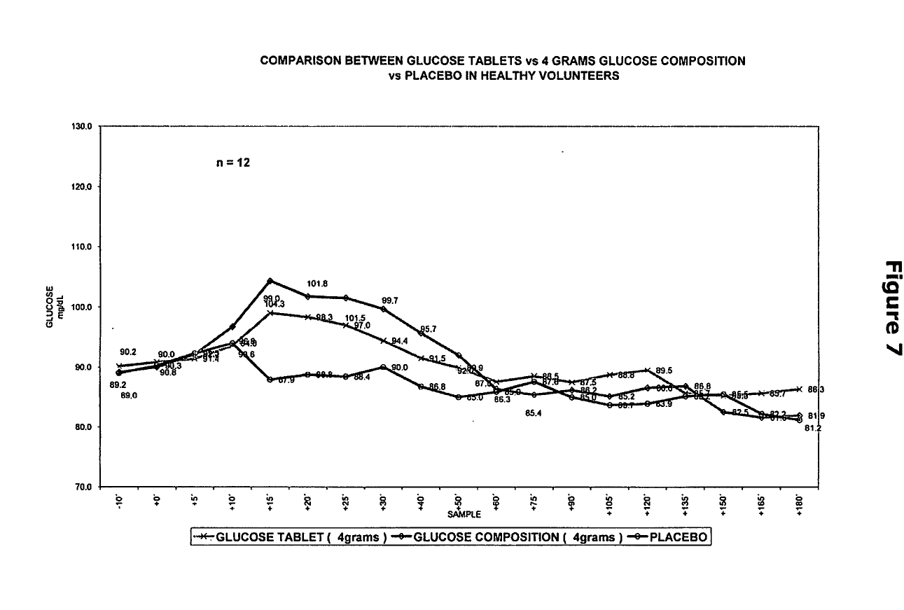

The mean blood glucose level values are summarized below and plotted in FIG. 7.

COMPARISON BETWEEN GLUCOSE TABLETS vs. 4 GRAMS GLUCOSE
COMPOSITION vs. PLACEBO IN HEALTHY VOLUNTEERS
Mean

| | 1 | 2 | 3 | 4 | 5 | 6 | 7 | 8 | 9 |
|---|---|---|---|---|---|---|---|---|---|
| | −10′ | +0′ | +5′ | +10′ | +15′ | +20′ | +25′ | +30′ | +40′ |
| Glucose | 89.2 | 90.0 | 92.2 | 96.8 | 104.3 | 101.8 | 101.5 | 99.7 | 95.7 |
| Composition | | | | | | | | | |
| (4 grams) | | | | | | | | | |
| SD | 3.1 | 3.0 | 3.5 | 5.4 | 8.6 | 7.7 | 5.3 | 3.6 | 4.7 |

| | 10 | 11 | 12 | 13 | 14 | 15 | 16 | 17 | 18 | 19 |
|---|---|---|---|---|---|---|---|---|---|---|
| | +50′ | +60′ | +75′ | +90′ | +105′ | +120′ | +135′ | +150′ | +165′ | +180′ |
| Glucose | 92.0 | 86.3 | 85.4 | 86.2 | 85.2 | 86.6 | 86.8 | 82.5 | 81.6 | 81.9 |
| Composition | | | | | | | | | | |
| (4 grams) | | | | | | | | | | |
| SD | 7.2 | 4.6 | 3.8 | 2.7 | 3.4 | 4.2 | 4.2 | 3.4 | 3.3 | 3.6 |

| | 1 | 2 | 3 | 4 | 5 | 6 | 7 | 8 | 9 |
|---|---|---|---|---|---|---|---|---|---|
| | −10′ | +0 ′ | +5′ | +10′ | +15′ | +20′ | +25′ | +30′ | +40′ |
| Glucose | 90.2 | 90.8 | 91.4 | 93.6 | 99.0 | 98.3 | 97.0 | 94.4 | 91.5 |
| Tablet | | | | | | | | | |
| (4 grams) | | | | | | | | | |
| SD | 4.6 | 4.5 | 4.1 | 6.1 | 7.9 | 7.9 | 7.7 | 8.0 | 6.6 |

| | 10 | 11 | 12 | 13 | 14 | 15 | 16 | 17 | 18 | 19 |
|---|---|---|---|---|---|---|---|---|---|---|
| | +50′ | +60′ | +75′ | +90′ | +105′ | +120′ | +135′ | +150′ | +165′ | +180′ |
| Glucose | 89.9 | 87.6 | 88.5 | 87.5 | 88.8 | 89.5 | 85.7 | 85.3 | 85.7 | 86.3 |
| Tablet | | | | | | | | | | |
| (4 grams) | | | | | | | | | | |
| SD | 5.5 | 3.2 | 3.9 | 3.2 | 3.9 | 4.6 | 4.2 | 4.5 | 4.1 | 4.1 |

| | 1 | 2 | 3 | 4 | 5 | 6 | 7 | 8 | 9 |
|---|---|---|---|---|---|---|---|---|---|
| | −10′ | +0 ′ | +5′ | +10′ | +15′ | +20′ | +25′ | +30′ | +40′ |
| PLACEBO | 89.00 | 90.25 | 92.33 | 94.00 | 87.92 | 88.75 | 88.42 | 90.00 | 86.75 |
| SD | 4.8 | 4.0 | 3.0 | 5.8 | 4.0 | 4.4 | 4.8 | 5.2 | 5.4 |

| | 10 | 11 | 12 | 13 | 14 | 15 | 16 | 17 | 18 | 19 |
|---|---|---|---|---|---|---|---|---|---|---|
| | +50′ | +60′ | +75′ | +90′ | +105′ | +120′ | +135′ | +150′ | +165′ | +180′ |
| PLACEBO | 85.00 | 85.92 | 87.58 | 85.00 | 83.67 | 83.92 | 85.17 | 85.50 | 82.17 | 81.17 |
| SD | 6.3 | 6.8 | 7.2 | 7.0 | 5.9 | 6.6 | 6.8 | 5.9 | 7.1 | 6.2 |

SD = standard deviation

FIG. 7 shows that the present inventive glucose composition is comparable to or better than glucose tablets in terms of its induced glucodynamic response.

The foregoing description is by way of example only and shall not be construed to limit the scope of the invention as defined by the following claims.

1. A glucose composition for delivering glucose across oral mucosa to raise the blood glucose level of an individual, the composition comprising:
a. an effective amount of glucose;
b. an effective amount of sodium glycocholate, and
c. an effective amount of a pharmaceutically acceptable carrier, wherein the composition is free of any additional active pharmaceutical agents.

2. The composition of claim 1, wherein the oral mucosa is the buccal mucosa.

3. The composition of claim 1, wherein the glucose is present in a concentration of from 10 to 80 w/w %.

4. The composition of claim 1, wherein the glucose is present in a concentration of from 20 to 70 w/w %.

5. The composition of claim 1, wherein the glucose is present in a concentration of from 30 to 60 w/w %.

6. The composition of claim 1, wherein each absorption enhancer is present in a concentration of from 0.01 to 5 w/w %.

7. The composition of claim 6, wherein each absorption enhancer is present in a concentration of from 0.01 to 3 w/w %.

8. The composition of claim 7, wherein each absorption enhancer is present in a concentration of from 0.01 to 1 w/w %.

9. The composition of claim 1, wherein the total concentration of absorption enhancer is less than 10 w/w %.

10. The composition of claim 9, wherein the total concentration of absorption enhancer is less than 5 w/w %.

11. (canceled)

12. The composition of claim 1, comprising glycerin.

13. The composition of claim 1, comprising sodium lauryl sulfate.

14. The composition of claim 1, comprising polyoxyethylene sorbitan monooleate.

15. The composition of claim 1, wherein the carrier is an aqueous solvent.

16. The composition of claim 1, further comprising at least one additional ingredient chosen from flavouring agents, colouring agents, preservatives and antimicrobial agents.

17. The composition of claim 16, comprising sodium benzoate as a preservative or antimicrobial agent.

18. The composition of claim 1, wherein the carrier is a chewing gum base.

19. A non-aerosol dispenser containing the composition of claim 1.

20. The non-aerosol dispenser of claim 19, wherein the dispenser is a metered dose dispenser.

21. The non-aerosol dispenser of claim 19, wherein the dispenser is adapted to deliver the composition as droplets having a size between 1 and 200 μm.

22. The dispenser of claim 21, wherein the dispenser is adapted to deliver the composition as droplets having a size between 1 and 20 μm.

23. The dispenser of claim 19, wherein the dispenser is adapted to dispense between 0.05 and 0.5 mL of the glucose composition per actuation.

24. The dispenser of claim 23, wherein the dispenser is adapted to dispense between 0.05 and 0.3 mL of the glucose composition per actuation.

25. The dispenser of claim 24, wherein the dispenser is adapted to dispense between 0.05 and 0.1 mL of the glucose composition per actuation.

26. A method of raising the blood glucose level of an individual comprising administering an effective amount of the composition of claim 1 to an oral mucosa of the individual.

27. The method of claim 26, wherein the administration is to the buccal mucosa.

28. The method of claim 26, wherein the administration comprises spraying the composition into the mouth of the individual using a non-aerosol dispenser.

29. The method of claim 28, wherein the non-aerosol dispenser is a metered dose dispenser.

30. The method of claim 26, wherein 30 to 50 mg of glucose is administered per actuation of the dispenser.

31. The method of claim 26, wherein the total amount of glucose administered per dose is from 150 to 300 mg.

32. The method of claim 31, wherein the total amount of glucose administered per dose is from 175 to 200 mg.

33. The method of any claim 28, wherein three to eight spray actuations are administered per dosing session.

34. The method of any claim 28, wherein between 0.05 and 0.5 mL of the glucose composition is sprayed per actuation.

35. The method of claim 34 wherein between 0.05 and 0.3 mL of the glucose composition is sprayed per actuation.

36. The method of claim 35 wherein between 0.05 and 0.1 mL of the glucose composition is sprayed per actuation.

37.-40. (canceled)

41. The composition of claim 1, comprising glycerin.

42. The composition of claim 41, comprising sodium lauryl sulfate.

43. The composition of claim 42, comprising polyoxyethylene sorbitan monooleate.

44. The composition of claim 1, comprising an effective amount of at least one additional absorption enhancer chosen from glycerin, sodium lauryl sulfate, polyoxyethylene sorbitan monooleate, lecithin, hyaluronic acid, glycolic acid, lactic acid, chamomile extract, cucumber extract, oleic acid, linoleic acid, linolenic acid, monoolein, monooleates, monolaurates, borage oil, evening primrose oil, menthol, trihydroxy oxo cholanyl glycine, polyglycerin, lysine, polylysine, triolein, polyoxyethylene ethers, polidocanol alkyl ethers, chenodeoxycholate, deoxycholate, alkali metal salicylate, pharmaceutically acceptable edetate, and pharmaceutically acceptable salts and analogues thereof

45. The composition of claim 44, further comprising at least one additional ingredient chosen from flavouring agents, colouring agents, preservatives and antimicrobial agents.

46. The composition of claim 45, comprising sodium benzoate as a preservative or antimicrobial agent.
